(19) United States
(12) Patent Application Publication
(10) Pub. No.: US 2025/0218567 A1
(43) Pub. Date: Jul. 3, 2025

(54) Meal plan generating method, apparatus, and computer implemented algorithm thereof

(71) Applicant: NUTRICIA EARLY LIFE NUTRITION (SHANGHAI) CO., LTD., Shanghai (CN)

(72) Inventors: Gregg Ward, Shanghai (CN); Mengjin Liu, Shanghai (CN); Bingzhi Guo, Shanghai (CN); Qiwen Song, Shanghai (CN)

(73) Assignee: NUTRICIA EARLY LIFE NUTRITION (SHANGHAI) CO., LTD., Shanghai (CN)

(21) Appl. No.: 19/084,817

(22) Filed: Mar. 20, 2025

Related U.S. Application Data
(63) Continuation of application No. PCT/CN2023/120713, filed on Sep. 22, 2023.

(30) Foreign Application Priority Data
Sep. 22, 2022 (WO) PCT/CN2022/120500

(51) Int. Cl. G16H 20/60

(52) U.S. Cl. CPC G16H 20/60

(57) ABSTRACT

The present invention may comprise a method, performed by an electronic device, for suggesting meal plans, comprising, determining a first element in the first meal plan to be replaced and a first nutritional vector of the first element, obtaining a candidate set of elements, obtaining candidate nutritional vectors for some or all the candidate elements in the candidate set, determining each similarity score based on each obtained candidate nutritional vector and the first nutritional vector, obtaining a second meal plan based on a second element which has a highest similarly score, wherein the first element in the first meal plan is replaced by the second element to form the second meal plan.

### FIELD OF THE INVENTION

The present invention relates to a meal plan generating method, apparatus, and computer implemented algorithm thereof.

### BACKGROUND

Nutrition is a critical driver of health and well-being of a person, which are normally obtained by the person from intakes of food (including drinks). For certain group of people, the nutrition intakes are very critical, e.g., for pregnant or lactating females, people with nutrition related diseases (e.g., diabetes), and specific segments such as senior/old people having special nutrition intake needs, etc. Therefore, it is very important to control/personalize the nutrition intakes according to the different situation of each targeted person.

The present invention provides a meal plan generating method, apparatus, and computer implemented algorithm thereof, which aims to optimize and personalize nutrition intakes and at the same time considers the preference of the users.

The present invention also provides a method to find a substitute meal plan for an existing meal plan.

### SUMMARY OF THE INVENTION

The present invention relates to a meal plan generating method, apparatus, and computer implemented algorithm/method thereof.

The present invention aims at providing personalized meal plans for users. The present invention is according to the claims.

### BRIEF DESCRIPTION OF THE DRAWINGS

The present invention will be discussed in more detail below, with reference to the attached drawings, in which:

FIG. 1A shows a meal plan generating method.

FIG. 1B shows a meal plan generating method.

FIG. 1C show a method for changing a suggested meal plan.

FIG. 1D shows a method for determining a substitute meal plan.

FIG. 2 shows an example for the evolution algorithm when generating the second subset.

FIG. 3 shows an example for determining weight values.

FIG. 4 shows an apparatus configured to perform a part of or all steps in the present invention.

### DESCRIPTION OF EMBODIMENTS

Embodiments of the present disclosure will be described herein below with reference to the accompanying drawings. However, the embodiments of the present disclosure are not limited to the specific embodiments and should be construed as including all modifications, changes, equivalent devices and methods, and/or alternative embodiments of the present disclosure.

The terms “have,” “may have,” “include,” and “may include” as used herein indicate the presence of corresponding features (for example, elements such as numerical values, functions, operations, or parts), and do not preclude the presence of additional features.

The terms “A or B,” “at least one of A or/and B,” or “one or more of A or/and B” as used herein include all possible combinations of items enumerated with them. For example, “A or B,” “at least one of A and B,” or “at least one of A or B” means (1) including at least one A, (2) including at least one B, or (3) including both at least one A and at least one B.

The terms such as “first” and “second” as used herein may modify various elements regardless of an order and/or importance of the corresponding elements, and do not limit the corresponding elements. These terms may be used for the purpose of distinguishing one element from another element. For example, a first printing form and a second printing form may indicate different printing forms regardless of the order or importance. For example, a first element may be referred to as a second element without departing from the scope the present invention, and similarly, a second element may be referred to as a first element.

It will be understood that, when an element (for example, a first element) is “(operatively or communicatively) coupled with/to” or “connected to” another element (for example, a second element), the element may be directly coupled with/to another element, and there may be an intervening element (for example, a third element) between the element and another element. To the contrary, it will be understood that, when an element (for example, a first element) is “directly coupled with/to” or “directly connected to” another element (for example, a second element), there is no intervening element (for example, a third element) between the element and another element.

The expression “configured to (or set to)” as used herein may be used interchangeably with “suitable for,” “having the capacity to,” “designed to,” “adapted to,” “made to,” or “capable of” according to a context. The term “configured to (set to)” does not necessarily mean “specifically designed to” in a hardware level. Instead, the expression “apparatus configured to . . . ” may mean that the apparatus is “capable of . . . ” along with other devices or parts in a certain context.

The terms used in describing the various embodiments of the present disclosure are for the purpose of describing particular embodiments and are not intended to limit the present disclosure. As used herein, the singular forms are intended to include the plural forms as well, unless the context clearly indicates otherwise. All of the terms used herein including technical or scientific terms have the same meanings as those generally understood by an ordinary skilled person in the related art unless they are defined otherwise. The terms defined in a generally used dictionary should be interpreted as having the same or similar meanings as the contextual meanings of the relevant technology and should not be interpreted as having ideal or exaggerated meanings unless they are clearly defined herein. According to circumstances, even the terms defined in this disclosure should not be interpreted as excluding the embodiments of the present disclosure.

The present invention provides a meal plan generating method, apparatus, and computer implemented algorithm thereof, which aims to optimize and personalize nutrition intakes and at the same time considers the preference of the users. The targeted user/person may be a pregnant female, a pregnant female having diabetes, a male, a baby, a child, a senior person, or any other persons. For example, the meal plan generating method may be for females in different stages, e.g., who are preparing for pregnancy, pregnant, recovering from delivery, or lactating, especially because that females at different stage may have different living styles, personal food preferences, and health conditions which require personalized nutrients.

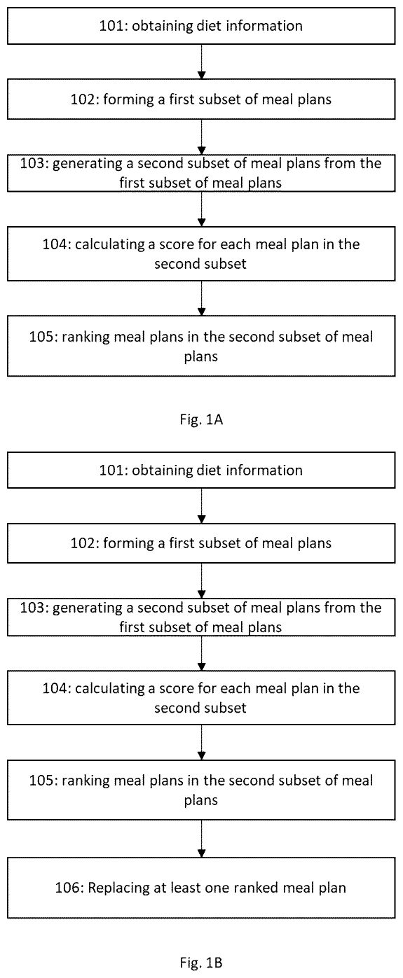

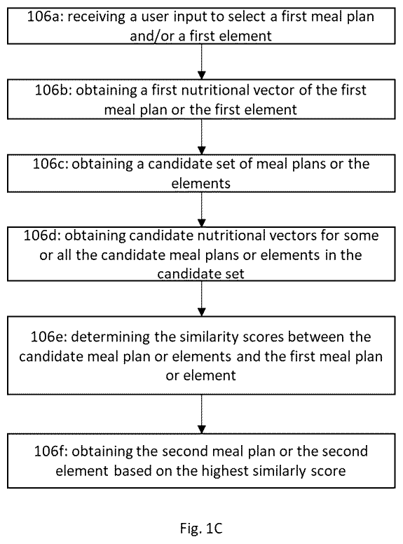

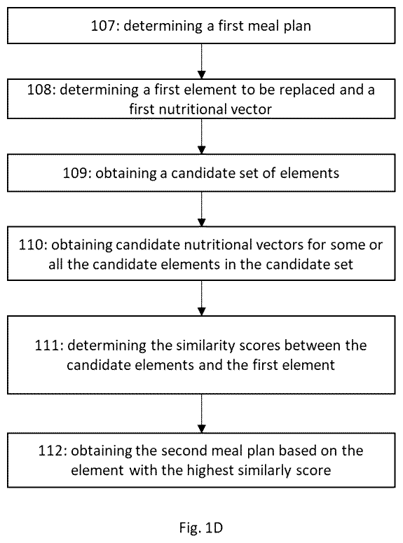

FIG. 1A shows a meal plan generating method.

In step 101, diet information of a user is obtained. The diet information may include any information relating to the diet of the user, for example, at least one of allergens, restricted ingredients, gene/DNA information, a number of meals for a day, height, weight, gender, active level, location information, diet preference, user pattern data, diet history, family history information, and stage of the user, for example the stage being pre-pregnancy, pregnancy, puerperium, or lactation. The diet information may further include other information, e.g., the health condition, diseases, body mass index (BMI), age, etc.

The user pattern data may comprise data of at least one of user behaviour information with respect to meal plans, e.g., recently selected (or any of saved, searched, commented, viewed longer than a predetermined period, etc.) meal plans or ingredients. The user pattern data may further include other user data when the user uses the present method to obtained meal plan suggestions, e.g., when using an application/software implementing the present method.

The location information may be the current location or the previous location of the user, e.g., the birth location, the longest stayed location in the past, etc.

The family history information may be relevant information of the family members of the user, e.g., a family member disease record, the weights of the family members, the diet preference of the family members, etc.

The user weight data of the user may comprise the current weight of the user, the weight change data of the user in the past periods, the user weight change data in connection to the meal plans, etc. The user weight change data can be very important since it gives hints on dynamic changes of the body/weights such that the meal plans can be suggested/scored accordingly. For example, a user who is losing weight too quickly may not be healthy, thus, meals suggested may include more energy/calories temporarily; or a user who is gaining weight very quickly, the energy in the suggested meals should be slowly reduced instead of immediate drop on calories.

The diet information may be obtained via at least one of the following ways, e.g., a questionnaire (e.g., on a smartphone/tablet), from an existing database (e.g., authorized medical records, recorded diet history, etc.), via certain devices (e.g., via a smart weigher for acquiring the weight of the use, via a device with a camera to estimate the BMI of the user), or any other ways.

In step 102, a first subset of meal plans is formed/generated based on the obtained diet information of the user. The first subset of meal plans comprises at least one meal plan.

A meal plan may be a plan comprising at least one element. For example, the element may be a collection of different food (the word “food” in this document contains both solid or mushy food, colloid, liquid/solution and drinks), for example, based on one or more food recipe (e.g., for one or more dishes). As another example, the one or more elements in each meal plan may comprise at least one of a breakfast menu, one or more staples for lunch, one or more staples for dinner, an extra meal menu, one or more dishes for lunch, and one or more dishes for dinner.

The first subset of meal plans may be formed by meal plans selected from an original set of meal plans (i.e., original meal plans, or a predetermined meal plan set, used interchangeably in this document). The original set of meal plans may be an original collection of all meal plans saved in a database (either internal or external the device comprising an application/software implementing the present method). For example, the first subset of meal plans may be selected from the original set based on the diet information directly, for instance via an artificial intelligent model. Or, the first subset of meal plans may be selected based on certain conditions, e.g., if the diet information indicates that the user is a senior person, then meal plans have liquid/soft food are more likely to be selected; if the diet information indicates that the user prefers spicy food, then meal plans with strong spicy flavour are more likely to be selected; if the diet information indicates that the user did not take any dairy in the past week, then meal plans with cheese and milk are more likely to be selected; if the diet information indicates that the user has an allergy to sea food, then meal plans with sea food will not be selected. Please note that the above conditions are only examples, which do not limit the scope prevent invention.

The first subset of meal plans may be formed from a filtered set of original meal plans (i.e., from the original set of meal plans), i.e., the original meal plans are first filtered by removing all the meal plans including allergens and/or restricted ingredients (based on the diet information) and/or removing meal plans including ingredients other than the existing meal preparing ingredients (i.e., the suggested meal plans may limited to the meal plans that are able to be prepared based on the existing food ingredients at home of the user); then meal plans are selected from the filtered original set when forming the first subset of meal plans (i.e., by any of the methods disclosed above). For example, the forming of the first subset of meal plans may be by removing zero, one or more meal plans from the predetermined/original meal plan set which comprises allergens and/or restricted ingredients according to the diet information and/or removing meal plans including ingredients other than the existing meal preparing ingredients, i.e., the first subset may be the filtered set of original meal plans.

The first subset of meal plans may be formed based on estimated at least one intake target, where the intake target may be the standard or recommended intake amount (e.g., mass, volume, or according to any other measurement methods) during a certain period (e.g., a day, a week, a month, the whole pregnancy period, etc.). For example, before Step 102, at least one an intake target for each ingredient, e.g., each food category, each macronutrient, and each micronutrient, may be determined/estimated according to the diet information, wherein each food category, each macronutrient and each micronutrient may be predetermined. The food category may comprise at least one of grain/cereal, dairy, fruit, vegetable, soy products/nuts, sweets/sugar, water, meat, poultry, fish and other alternatives. The macronutrient may comprise at least one of energy, fat, protein, carbohydrate, and other macronutrients. The micronutrient may comprise at least one of calcium, Fe (iron/ferrum), zinc, folate, vitamin D, vitamin B12, vitamin B6, vitamin C, vitamin B2, Vitamin E, DHA (Docosahexaenoic acid), and other micronutrients. For example, the determining of the intake target may be further based on standard nutrient recommendations according to the diet information of the user. The determining of the intake target may be further based on other information, e.g., the biometric information, the microbiome information and/or blood glucose information of the user.

Each intake target, for each food category, each macronutrient, or each micronutrient, may be determined according to the obtained diet information. For example, if the diet information indicates that the user is pregnant which may request the total energy intake per day within a certain range, then meal plans within this range have a higher chance to be selected when forming the first subset; if the diet information indicates that the user is lactating which may request the folate intake per day within a range, then meal plans within this folate intake range have a higher chance to be selected when forming the first subset; etc. The database may be formed as at least one lookup table which provides cross references between different diet information and standard intake targets for different ingredients.

For example, each intake target may be determined by comparing the diet information with a standard reference database (e.g., look up tables). For example, a standard reference database links the average/standard intake for each ingredient (e.g., each food category, each macronutrient, and/or each micronutrient) to the specific diet information. For example, the database may indicates how much is the average/standard energy intake per day for a pregnant female with certain body weight, age and heath conditions; how much is the average/standard vitamin D intake within a day for a senior person at a certain age.

The above determining of the intake targets may be omitted in the method, or, in addition, may be performed before calculating the score for each meal plan in step 104, or during step 103 when using the evolution algorithm, which will be discussed later in this document.

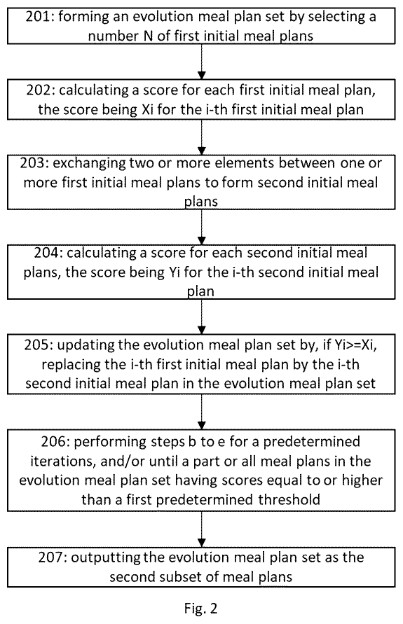

In step 103, a second subset of meal plans is generated from the first subset of meal plans, e.g., via an evolution algorithm, or a trained artificial intelligence model. An example of using an evolution algorithm will be presented in FIG. 2 later.

In step 104, a score for each meal plan in the second subset is calculated. The score of a meal plan may be calculated based on at least two score parts, wherein a score part may be an ingredient score, a weekly score, a daily score, a gene score or a preference score. The higher scores may be defined as more desirable, or the lower scores may be defined as more desirable. For the purpose of illustration, in the remaining content of this document, we assume a higher score is more desirable, however, the option for a lower score being more desirable is also implicitly disclosed.

The ingredient score may be calculated based on at least one of a total food category score, a total macronutrient score, and a total micronutrient score. The total food category score may be a sum of individual food category scores, and an individual food category score may be calculated according to the amount (mass, volume/or other measurement methods) of a food category comprised in the meal plan and intake target of the food category, for example, the food category being at least one of grain, cereal, diary, fruit, vegetable, soy products, nuts, sweets, water, meat, fish and alternatives. The total macronutrient score is a sum of individual macronutrient scores, and an individual macronutrient score may be obtained according to the amount (mass, volume/or other measurement methods) of a macronutrient comprised in the meal plan and intake target of the macronutrient. The total micronutrient score may be a sum of individual micronutrient scores, and an individual micronutrient score may be obtained according to the amount (mass, volume/or other measurement methods) of a micronutrient comprised in the meal plan and intake target of the micronutrient.

An intake target for each food category, each macronutrient, and each micronutrient, may be determined according to the diet information, which has been presented above and will not be repeated here. An individual food category score may be based on the determined intake target of this individual food category; An individual macronutrient score may be based on the determined intake target of this individual macronutrient; An individual micronutrient score may be based on the determined intake target of this individual micronutrient. A higher (e.g., more desirable) individual ingredient score (i.e., for any of food category, macronutrient and micronutrient) may be given to the individual ingredient (i.e., for any of food category, macronutrient and micronutrient) if the amount (e.g., mass, volume, or according to any other measurement methods) of said ingredient contained in a meal plan is closer to the intake target of said ingredient. When the amount of the ingredient contained in the meal plan is further from the intake target of said ingredient, the individual ingredient score is lower (e.g., less desirable). Some examples on how to determine the individual ingredient score are shown below.

An example of an individual food category score table for grain and cereal intake (grams per day) is shown in Table 1.

TABLE 1
Example for grain and cereal score

| | Diet information vs | | | | |
|---|---|---|---|---|---|
| | grain and cereal score | | | | |
| | 1 | 2 | 3 | 2 | 1 |
| Early pregnancy | <200 | 200-249 | 250-300 | 301-350 | >350 |
| Mid pregnancy | <225 | 225-274 | 275-325 | 326-375 | >375 |
| Late pregnancy | <250 | 250-299 | 300-350 | 351-400 | >400 |
| Lactating phase | <250 | 250-299 | 300-350 | 351-400 | >400 |
| Other adults | <200 | 200-249 | 250-300 | 301-350 | >350 |

As shown in Table 1, for an early pregnant female, if the intake of grain and cereal during a day is less than 200 grams, the grain and cereal score (as an example of one of the individual food category scores) is 1; if it is 200 to 249 grams, the grain and cereal score is 2; if it is 250 to 300 grams, the grain and cereal score is 3 (most desirable/most healthy according to the determined intake target for grain and cereal); if it is 301 to 350 grams, the grain and cereal score drops to 2 (too much already); if it is more than 350, the grain and cereal score is 1. For each of the food category, such a score may be calculated. In this example table, the intake targets of grain and cereal (i.e., standard or recommended grain and cereal intake amount per day) according to different diet information (early pregnancy, mid pregnancy, late pregnancy, lactating phase and other adults) are given in the column when the grain and cereal score is the highest (3).

An example of an individual macronutrient score table for fat intake (grams per day) is shown in Table 2.

TABLE 2
Example for fat score

| | Diet information vs fat score | | | | |
|---|---|---|---|---|---|
| | 1 | 2 | 3 | 2 | 1 |
| Preparing for | <40 | 40-44 | 45-55 | 56-60 | >60 |
| pregnancy | | | | | |
| Early pregnancy | <40 | 40-44 | 45-55 | 56-60 | >60 |
| Mid pregnancy | <47.2 | 47.2-52.1 | 53.1-64.9 | 65.9-70.8 | >70.8 |
| Late pregnancy | <50.4 | 50.4-55.7 | 56.7-69.3 | 70.3-75.6 | >75.6 |
| Lactating phase | <51.2 | 51.2-56.6 | 57.6-70.4 | 71.4-76.8 | >76.8 |
| Other adults | <40 | 40-44 | 45-55 | 56-60 | >60 |

As shown in Table 2, for an early pregnant female, if the intake of fat during a day is less than 40 grams, the fat score (as an example of one of the individual macronutrient scores) is 1; if it is 40 to 44 grams, the fat score is 2; if it is 45 to 55 grams, the fat score is 3 (most desirable/most healthy); if it is 56 to 60 grams, the fat score drops to 2 (too much already); if it is more than 60, the fat score is 1. For each of the macronutrient, such a score may be calculated. In this example table, the intake targets of fat (i.e., standard or recommended fat intake amount per day) according to different diet information (Preparing for pregnancy, early pregnancy, mid pregnancy, late pregnancy, lactating phase and other adults) are given in the column when the fat score is the highest (3).

An individual micronutrient score may be determined similarly as in Table 1 and Table 2 for the food category and macronutrient, which will not be repeated here.

The total food category score may be defined as an average score of the at least one of the individual food category scores. For example, if eight different food categories are considered, then Food Category Score=(Grain and Cereal Score+dairy Score+Fruit Score+Vegetable Score+Soy products and nuts Score+Sweets Score+Water Score+Meat, poultry fish and alternatives)/8. Alternatively, the total food category score may be defined in other ways, e.g., the lowest within the individual food category scores or highest within the individual food category scores, or any other ways.

Similarly, the total macronutrient score may be defined as an average score of at least one of the individual macronutrient scores; and the total micronutrient score may be defined as an average score of at least one of the individual micronutrient scores. Alternatively, the total macronutrient (or micronutrient) score may be defined in other ways, e.g., the lowest within the individual scores or highest within the individual scores, or any other ways.

The ingredient score (i.e., the total ingredient score) may be calculated according to at least one of a total food category score, a total macronutrient score, and a total micronutrient score. The ingredient score may be further adjusted to fit a certain scale, e.g., with 10 as the highest. For example, the ingredient score may be a sum of at least one of a total food category score, a total macronutrient score, and a total micronutrient score. Or after the sum, the value may be further adjusted. For example, if the maximal possible scores of the total food category score, the total macronutrient score, and the total micronutrient score are X, Y and Z and the total scores (i.e., real scores) for them are x, y and z. Then the ingredient score may be calculated as Wi*(x/X+y/Y+z/Z)/3 or (x/X+y/Y+z/Z)/3, where Wi is a weight given to the ingredient score when calculating the score of the meal plan. Other alternatives are also possible.

A weekly score may be determined according to at least one predetermined weekly rule. The at least one predetermined weekly rule may be determined according to the diet information, i.e., different diet information may correspond to different weekly rules. If a rule (from the at least one predetermined weekly rule) is met, then an individual rule score is assigned (e.g., an integer larger than 0), otherwise, another individual rule score is assigned (e.g., 0). The individual rule score may be further according to a predetermined weight for the corresponding rule score. An example of weekly rules and the weight for the early pregnancy stage (as an example of the diet information) is shown in Table 3.

TABLE 3
Example of weekly rules and weights for an early pregnancy stage.

| Weekly | | |
|---|---|---|
| rule index | Weekly Rules | Weight |
| 1 | Eat seafood 2-3 times a week | 2 |
| 2 | Eat high Fe contained dish 1-2 times a week | 3 |
| 3 | Eat high Iodine dish twice a week | 4 |
| 4 | Eat High DHA contained dish twice a week | 5 |

In the above example in Table 3, there are four weekly rules, and each rule has a different rule weight. If only Rule 1 is met, then, the weekly score (i.e., total) may be calculated as 4*2/(2+3+4+5)=0.57. E.g., if Rw(i) indicates whether Rule i is met or not by a meal plan, when Rw(i)=1, Rule i is met; when Rw(i)=0, Rule i is not met. The weight of each rule may be indicated as Ww(i). The total number of rules are Nw. As an example, the weekly score may be calculated as Nw*sum(Rw(i)*Ww(i))/sum(Ww(i)).

The daily score may be based on at least one daily rules concerning a one-day meal plan; the gene/DNA score may be based on gene rules concerning diet requirements according to gene/DAN of the user; the preference score may be based on preference rules concerning diet preferences of the user. The daily score, the gene/DNA score and the preference score may be calculated in the same way as for the weekly score.

Examples of the daily rules, gene rules and preference rules are shown in tables 4, 5, and 6 respectively.

TABLE 4
Example of daily rules and weights for an early pregnancy stage.

| Daily rule index | Daily Rules | Weight |
|---|---|---|
| 1 | Green vegetables >= 1 times | 2 |
| 2 | Soy products >= 1 times | 2 |
| 3 | Iodized salt contains 96-144 μg | 1 |
| 4 | calcium >= 1000 | 1 |

TABLE 5
Example of gene/DNA rules and weights
for an early pregnancy stage.

| Gene rule index | Gene Rules | Weight |
|---|---|---|
| 1 | Vitamin D requirements are high | 1 |
| 2 | Vitamin B6 requirements are high | 2 |

TABLE 6
Example of Preference rules and weights
for an early pregnancy stage.

| Preference | | |
|---|---|---|
| rule | | |
| index | Preference Rules | Weight |
| 1 | Taste preference: dishes that meet users' taste | 1 |
| | needs appear once a day | |
| 2 | Season preference: over 40% of dishes in a day | 2 |
| | meet users' seasonal preference | |
| 3 | Cooking type preference, dishes that meet the user's | 3 |
| | cooking method preference appear once a day | |
| 4 | Cuisine preference: dishes that meet the user's | 1 |
| | cuisine preference appear once a day | |

The daily score may be calculated in the same way as for the weekly score. Here the rules referred to are daily rules. For example, if Rd(i) indicates whether Rule i is met or not, when Rd(i)=1, Rule i is met; when Rd(i)=0, Rule i is not met. The weight of each rule may be indicated as Wd(i). The total number of rules are Nd. As an example, the daily score may be calculated as Nd*sum(Rd(i)*Wd(i))/sum(Wd(i)).

The gene/DNA score may be calculated in the same way as for the weekly score. Here the rules referred to are gene rules. For example, if Rg(i) indicates whether Rule i is met or not, when Rg(i)=1, Rule i is met; when Rg(i)=0, Rule i is not met. The weight of each rule may be indicated as Wg(i). The total number of rules are Ng. As an example, the gene score may be calculated as Ng*sum(Rg(i)*Wg(i))/sum(Wg(i)). As another example, rs1801133 from the gene/DNA information of a user may have one of types of GG, AG and AA, and folate needs/absorbing ability for different types are GG>AG>AA, which may be the basis for generating additional gene rules/scores.

The preference score may be calculated in the same way as for the weekly score. Here the rules referred to are preference rules. For example, if Rp(i) indicates whether Rule i is met or not, when Rp(i)=1, Rule i is met; when Rp(i)=0, Rule i is not met. The weight of each rule may be indicated as Wp(i). The total number of rules are Np. As an example, the preference score may be calculated as Np*sum(Rp(i)*Wp(i))/sum(Wp(i)).

A score of a meal plan may be calculated based on at least two score parts, and/or a score part being an ingredient score, a weekly score, a daily score, a gene score or a preference score. Weights may be assigned to each of the score part. For example, if the score for score part j is s (j) and the weight for score part j is w(j), then the meal plan score S may be calculated S=sum(s(j)*w(j)).

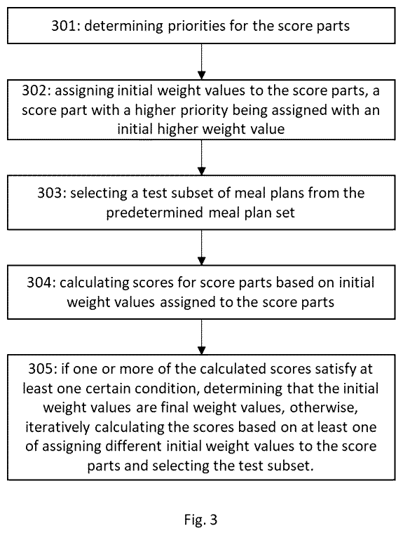

As disclosed above, when calculating the score of a meal plan, the weight values (w(j)) may be used. I.e., when calculating the score of the meal plan, each score part may be assigned with a weight value. The weight values (w(j)) for the score parts (j) may be determined by: determining priorities for the score parts; assigning initial weight values to the score parts, a score part with a higher priority being assigned with an initial higher weight value; selecting a test subset of meal plans from the predetermined meal plan set, e.g., a part of all meal plans in the predetermined/original meal plan set; calculating scores for score parts based on initial weight values assigned to the score parts; if one or more of the calculated scores satisfy at least one certain condition, determining that the initial weight values are final weight values, otherwise, iteratively assigning different initial weight values to the score parts and selecting the test subset and calculating the scores. An example of the determination of the weight value method for different score part is shown in FIG. 3.

In step 301, the priorities of the score parts may be determined. For example, if the score parts comprise an ingredient score, a weekly score, a daily score, a gene score or a preference score, the priorities of the score parts may be determined as ingredient score>-gene score>=weekly score>=daily score>=preference score, or another priority sequence may be determined.

In step 302, initial weight values to the score parts may be assigned, e.g., a score part with a higher priority being assigned with a higher initial weight value. For example, the initial weight values may be assigned as ingredient score (10)>=gene score (10)>=weekly score (10)>=daily score (10)>=preference score (8), where 10, 10, 10, 10 and 8 are the weight values respectively.

In step 303, a test subset of meal plans is formed from the predetermined meal plan set (i.e., the original set of meal plans or the original meal plans, used interchangeably in this document). For example, the total number of the meal plans in the test subset may be around 100 to 200 meal plans or even more.

In step 304, score parts in each of the meal plans in the test subset may be calculated. Then the score for each of the meal plans in the test subset may be calculated based on the initial weight values for the score parts. During the calculation of the score parts for each test meal plan, some test/predetermined diet information may be used. The test/predetermined diet information may be randomly generated or selected from an existing diet information database according to some predetermined conditions. Alternatively, the test/predetermined diet information may be the diet information of the current user (i.e., the individual ingredient scores and rule weights are based on the actual diet information of the current user), such that the weight values are personalized.

The weight values determined may be further personalized such that a meal plan may be given a score more accurately. For example, the test subset may be selected based on the diet information of the current user in step 303, e.g., in the same way as when selecting the first subset of meal plans in step 102. For example, the test subset may be the same as the first subset of meal plans in step 102, or a part of the first subset of meal plans in step 102. In this way, the weight values are further personalized according to the diet information of the current user.

In step 305, if one or more of the calculated scores satisfy at least one certain condition, determining that the initial weight values are final weight values for the score parts, otherwise, iteratively assigning different initial weight values to the score parts and selecting the test subset and calculating the scores. For example, the at least one certain condition may be that if the percentage, of the scores from all the test meal plans that are higher than a certain score threshold (e.g., 5, 5,5, 6, 6.5 or other scores), is higher than a certain percentage threshold, e.g., 50% or other percentages, then the initial weight values may be considered good enough and determined to be the final weight values. Another example of the certain condition may be that if the scores for all the test meal plans calculated based on the initial weight values are with a predetermined error range of predetermined reference scores, e.g., they are about the same as predicted by the corresponding scores given by the expert(s) based on the meal plans. “About the same” may mean that the score variations are within a certain range (e.g., 5% or 10%), and/or the distribution of the percentages of the scores within different score ranges (e.g., scores <2, between 2 and 4, between 4 and 8, >8) matches the prediction of the expert. The expert here may be a person or an artificial intelligence model that is trained to give scores to meal plans based on diet information.

In step 305, if the certain condition is not met, iteratively performing at least one of assigning different initial weight values to the score parts, selecting the test subset and calculating the scores, i.e., iteratively calculating the scores based on at least one of newly assigned initial weight values to the score parts and newly selected test subset. For example, the initial weight values may be adjusted but still meet the priority sequence, then the adjusted initial weight values may be used to calculating the scores. The adjustment of the weight values may be performed iteratively until the at least one certain condition is met, i.e., the adjusted weight values become the final weight values. During each iterative adjustment of the initial weight values, the test subset of meal plans may be iteratively reselected, alternatively the same test subset of meal plans may be used until the final weight values are determined. The reselecting of the test subset of meal plans may be based on the same method as the selecting of the test subset in step 303; in addition, the reselected test subset may overlap only partly with the test subset in previous iterations. The overlap part (i.e., overlapped meal plans) may be constrained to a maximal range in a new test subset, e.g., a certain percentage of the meal plans should be never included in the previous test subsets. This percentage may be 20%, 30%, 40%, 50%, 60%, 70%, 80% and other percentages. For example, if the at least one certain condition is not met, the initial weight values may be adjusted and a new test subset may be reselected, then the scores may be calculated based on the adjusted initial weight values and the new test subset; if the at least one certain condition is not met, the initial weight values may be adjusted and a new test subset may be reselected again, then the scores are calculated again; the iteration may end when the at least one certain conditions are met. In addition, the iteration may also end when the overall iteration times are more than a predefined number, e.g., 20 time, 50 times, 100 times, or even more.

Going back to FIG. 1A/1B, in step 104 the scores for the meal plans in the second subset may be calculated according to the above disclosed method.

In step 105, meal plans in the second subset of meal plans are ranked. In addition, a number of top ranked meal plans may be displayed/suggested to the user, e.g., top one, three or five ranked meal plans may be displayed and/or suggested to the user, either with or without the corresponding scores displayed. A user input may be received to select one of the top ranked meal plans. The meal plan with the highest score from the second subset may be suggested to the user and/or only this meal plan may be outputted to the user.

The method may further comprise a step of replacing at least one ranked meal plan as shown in step 106 of FIG. 1B. All other steps in FIG. 1B are the same as in FIG. 1A. For example, after step 105 where the second subset of meal plans are ranked and/or suggested, a user input may be received, which may be selecting and changing one or ranked and/or suggested meal plans in step 105. Then selected one or more meal plans may be replaced by other meal plans. Alternatively, the method may automatically select a ranked meal plan (and/or an element in a ranked meal plan) to be changed/replaced, e.g., based on one or more pre-determined conditions. For example, if the exact same meal plan has been suggested already in the last pre-determined number of days, the ranked meal plan includes an ingredient that is not available to the user or in the present season, or if a user has provided a further input with updated personal preference or updated health conditions, etc., then such meal plans will be suggested to be replaced automatically by the device.

For example, an input may be received from the user to change a first meal plan within the top ranked meal plans and then suggest a second meal plan based on the first meal plan. In addition, or alternatively, a user may be able to select and change one or more elements in the suggested meal plan in step 105. Then selected one or more elements may be replaced by elements. I.e., an input may be received from the user to change a staple element (e.g., bread) with another staple element (e.g., bread). By replacing the selected element with a different element (but similar), a second meal plan may be formed.

The second meal plan may be determined based on at least one of one or more properties of the first meal plan (e.g., similarity between the two meal plans), the diet information (e.g., diet preference, user pattern data, etc.), some general rules obtained from a group of users (e.g., milk in the breakfast may be replaced by fried eggs, bread may be used to replace potatoes, etc.), and meal plans preferred by other similar users (e.g., if two user are determined to be similar, the replacement meal plan may be from the preferred meal plans of the other user). If the user only selects to replace one or more elements in the first meal plan, the forming of the second meal plan may be further based on at least a similarity between the at least one element to be replaced and at least one different element to replace it, and the diet information. For example, similar staple elements may replace each other, or similar dairy products may replace each other when forming the second meal plan.

For example, the user pattern data in the diet information may comprise data of at least one of user behaviour information with respect to meal plans, which may be used to select the second meal plan as a replacement of the first meal plan. As another example, the diet preference in the diet information may be used to select the second meal plan.

A similarity between meal plans may be determined based on at least one of the total weights of the meals, comprised ingredients, proportions of the ingredients, foot category compositions, macro ingredient compositions, micro ingredient compositions, total calories, etc.

Furthermore, within step 106 of replacing of the first meal plan in the ranked meal plans, a first element in the first meal plan may be replaced by a second element from an element candidate set to form the second meal plan. A similarity score between two elements may be calculated based on respective nutritional vectors of the two elements, and the second element may have the highest similarity score with the first element amongst elements in the element candidate set.

Instead of replacing one (or more) element, the meal plan may be replaced as a whole. For example, in step 106, the first meal plan to be replaced may be analysed and a further searching of the replacement meal plan may performed in the predetermined meal plan set, the first subset of meal plans or the second meal plans. During the searching of the second meal plan, a similarity between the meal plans may be analysed. Then, a meal plan which is the most similar (e.g., with the highest similarity score) to the first meal plan is outputted as the second meal plan (i.e., the substitute meal plan).

For example, when determining the similarity score between two meal plans or two elements, analyzing of the first meal plan or element may be performed, which may comprise obtaining of a nutritional vector for each meal plan or element. A nutritional vector may comprise a plurality of values and each value may reflect a nutritional aspect (or dimension) of the meal plan or element. For example, a nutritional vector (of a meal plan or an element of a meal plan) may include a vector comprising values reflecting at least one of the food categories, various macronutrient compositions (by weight or volume), and various micronutrient compositions (by weight or volume). One preferred example of the nutritional vector may comprise values from four dimensions, namely <total energy, total protein, total fat, total carbohydrate>. The values may be weight, volume, weight percentages, or volume percentages of the corresponding dimensions.

A similarity between meal plans (or two elements) may be determined based on the similarity score. The similarity score may be calculated based on the nutritional vectors of the corresponding meal plans (or elements). The similarity score may be between [0, 1], wherein a larger the value may indicate a higher similar level. For example, 1 may represent that the two meal plans (or elements) are the same.

For example, a similarity score between meal plan (or element) a and meal plan (or element) b may be determined according to the nutritional vector A and nutritional vector B corresponding to meal plan (or element) a and b respectively. An example for the calculation may be according to the below equation:

$$S ( A , B ) = \frac{A \cdot B}{‖ A ‖ ‖ B ‖} = \frac{{\sum }_{i = 1}^{n} {A}_{i} {B}_{i}}{\sqrt{{\sum }_{i = 1}^{n} {A}_{i}^{2}} \sqrt{{\sum }_{i = 1}^{n} {B}_{i}^{2}}}$$

wherein A is <A₁, A₂, . . . Aₙ> and B is <B₁, B₂, . . . Bₙ>. It can be seen from the above equation that it calculates cos(θ) wherein θ is the angle between the vectors of A and B, i.e., when θ=0, the two vectors are the same, i.e., the similarity is the highest of 1; when θ=π/2, the two vectors are orthogonal to each other, i.e., the similarity is the lowest of 0.

To be more specific, within step 106 the replacement may be on the element level, not at the meal plan level. For example, when replacing the at least on ranked meal plan, a user input may be received to replace one or more elements (i.e., one or more of the first elements) within the to-be-replaced meal plan (i.e., the first meal plan). The searching for one or more substitute element is similar to the above searching of the replacement meal plan. For example, first, each of the to-be-replaced elements (i.e., the first elements) may be analysed to generate a nutritional vector A, and a candidate replacement element from a candidate set may be analysed to generate a nutritional vector B. Then a similarity score between the to-be-replaced element and the candidate replacement elements may be calculated based on the nutritional vectors A and B in the same way as shown in the above equation. After all the candidate elements are compared with the to-be-replaced element, the most similar candidate element (i.e., the second element, which may have the highest similarity score) is used to replace the to-be-replaced element (i.e., the first element) in the to-be-replaced meal plan (i.e., the first meal plan) to generate the replacement meal plan (i.e., the second meal plan).

The above changing of the meal plan step in step 106 is summarized in FIG. 1C. In step 106a, a user input may be received, and the user input may select one or more first meal plans, and/or one or more first elements to be replaced from the ranked/suggested meal plans. This step may be omitted, since the selection may be automatically performed by the electronic device, for example, the first meal plan and/or the first element may be determined according to at least one predetermined condition, and the predetermined condition may include at least one of that if a same meal plan and/or element has been suggested in the last predetermined number of days, if ingredient in a meal plan and/or element is not available, or if a further user input is received which conflicts with the meal plan and/or element, then such meal plans will be determined to be changed/replaced. The further user input may include updated diet information, which may include at least one of allergens, restricted ingredients, gene/DNA information, a number of meals for a day, height, weight, gender, active level, location information, diet preference, diet history data, user pattern data, family history information, and stage of the user, for example the stage being pre-pregnancy, pregnancy, puerperium, or lactation.

The selection may even be randomly determined by the electronic device in order to purposely diversify the suggested meal plans and/or elements.

In step 106b, a first nutritional vector is obtained for the first meal plan or first element. The nutritional vector of a meal plan or an element may be a vector comprising a plurality of values and each value may reflect a nutritional aspect (or dimension) of the meal plan or element. For example, a nutritional vector may be a vector comprising values reflecting at least one of the food categories, various macronutrient compositions (by weight or volume), and various micronutrient compositions (by weight or volume). One preferred example of the nutritional vector may be a vector comprising values from four dimensions, namely <total energy, total protein, total fat, total carbohydrate>. The values may be weight, volume, weight percentages, or volume percentages of the corresponding dimensions.

The nutritional vector can represent the nutritional information of a meal plan or element, which may be further used to compare different meal plans or different elements (e.g., obtaining similarity levels between them). The nutritional vector may be predetermined and stored in a memory of the electronic device, or received from an external device.

In step 106c, a candidate set of meal plans for elements is obtained. The candidate set defines the search space including candidates for the second meal plan or the element. For example, the candidate set for meal plans may be one of the predetermined meal plan set, the first subset of meal plans or the second meal plans. The element candidate set may be a set including all the elements within the same type of elements (i.e., belonging to the same type as the first element) within one of the predetermined meal plan set, the first subset of meal plans or the second meal plans. The element type may be at least one type of a breakfast menu, one or more staples for lunch, one or more staples for dinner, an extra meal menu, one or more dishes for lunch, and one or more dishes for dinner.

When the candidate set is ready in step 106c, all or some of the candidates will be compared with the first meal plan (or the first element) and the most similar candidate will be determined. For example, not all the candidates may need to be searched because some of them may be pre-filtered out according to some predetermined rules, e.g., updated diet information in the further user input, which may include at least one of allergens, restricted ingredients, gene/DNA information, a number of meals for a day, height, weight, gender, active level, location information, diet preference, diet history data, user pattern data, family history information, and stage of the user, for example the stage being pre-pregnancy, pregnancy, puerperium, or lactation.

When comparing a candidate meal plan or element with the first meal plan or the first element, in step 106d, a candidate nutritional vector of the candidate meal plan or element is obtained.

In step 106e, each similarity score is calculated between each candidate meal plan or element and the first meal plan or element. A similarity score may be calculated based on corresponding nutritional vector of the candidate and the nutritional vector of the first meal plan or element. One example of the calculation has been presented above already, which will not be repeated here.

In step 106f, the candidate with the highest similarity score to the first meal plan or the first element is selected, i.e., as the second meal plan or second element. If only the element is exchanged (i.e., not the whole first meal plan), the second element may be used to replace the selected element within the first meal plan to form the second meal plan. The second meal plan may be notified to the user.

In the above meal plan changing method, as a preferred embodiment, a suggested/ranked meal plan may be first determined as the first meal plan and then an element within this meal plan is determined (as the first element) to be changed, where in this embodiment only the element is changed to the second element when forming the second meal plan, but not the whole first meal plan.

FIG. 1D shows a method for determining a substitute meal plan, which is similar as the method in FIG. 1C.

In step 107, a first meal is determined which may include one or more elements. For example, a user may input the meal plan directly to the electronic device, the user may select the meal plan from the suggested meal plans in FIGS. 1A and 1B (e.g., as in step 106a), or the first meal plan may be randomly selected from a predetermined meal plan set by the device.

In step 108, a first element in the first meal plan is determined to be replaced. This may be according to a further selection input of a user or a random selection of the device. After the first element is determined, a first nutritional vector of the first element is determined as in step 106b.

In steps 107 and 108, the first meal plan and/or the first element may be determined according to user inputs, or may be determined according to at least one predetermined condition. The predetermined condition may include at least one of that if a same meal plan and/or element has been suggested in the last predetermined number of days, if ingredient in a meal plan and/or element is not available, and if a further user input is received which conflicts with the meal plan and/or element, then such meal plans and/or elements will be determined to be replaced (as the second meal plan or the second element). The further user input may include updated diet information.

In step 109, a candidate set of elements is obtained (the same as in 106d), e.g., according to element types. The element type may be at least one type of a breakfast menu, one or more staples for lunch, one or more staples for dinner, an extra meal menu, one or more dishes for lunch, and one or more dishes for dinner. When obtaining the candidate set, some predetermined filtering may be performed according to the diet information or the updated diet information.

In step 110, nutritional vectors for some or all the candidate elements in the candidate set may be obtained (the same as in step 106d).

In step 111, the similarity scores between the candidate elements and the first elements may be determined based on their corresponding nutritional vectors (the same as in step 106e).

In step 112, the candidate element with the highest similarity score is selected as the second element, and it may be used to replace the first element in the first meal plan, such that a second meal plan (i.e., the substitute meal plan) is formed (as in one alternative of step 106f).

The method in FIGS. 1C and 1D may be performed iteratively. For example, if the user is still not satisfied with the suggested meal plan, a further change input and selection of the first meal plan (and/or the first element) may be received, such a new second meal plan can be suggested.

In the method of FIG. 1A/1B, step 102 forming of the first subset of meal plans and/or step 103 generating of the second subset of meal plans may be performed iteratively until a part or all of meal plans in the second subset of meal plans have scores (i.e., calculated by step 104) higher than or equal to a second predetermined threshold. For example, the first subset of meal plans and/or the second subset of meal plans may be regenerated until the at least some meal plans in the second subset is good enough for the user. This guarantees that the suggested meal plan higher or equal to at least the threshold score.

If step 102 the forming of the first subset of meal plans and step 103 generating of the second subset of meal plans are performed more than predetermined iterations, step 105 the selecting of the meal plan from the second subset of meal plans may be performed even if none in the second subset of meal plans has a score higher than or equal to the second predetermined threshold. This may avoid endlessly iterations.

FIG. 2 shows an example of the evolution algorithm when generating the second subset in step 103.

In step 201, an evolution meal plan set may be formed by selecting a number N first initial meal plans. The N first initial meal plans may be randomly selected from the first subset of meal plans, or selected based on certain conditions, for example, selected based on the most frequently suggested meal plans, the least frequently suggested meal plans, the most popular meal plans based on survey of the users, etc.

In step 202, a score for each first initial meal plan may be calculated, where the score may be noted as Xi for the i-th first initial meal plan. The score calculating method may be the same calculating method as disclosed for step 104 in FIG. 1A/1B. In addition, if one, some or all the scores of Xi meet the condition equal to or higher than a first predetermined threshold, the first initial meal plan may be directly outputted as the second subset of the meal plans and the later steps in FIG. 2 may not be performed.

In step 203, one or more elements between two or more first initial meal plans may be exchanged to form second initial meal plans. The one or more elements in each meal plan may comprise at least one of a breakfast menu, one or more staples for lunch, one or more staples for dinner, an extra meal menu, one or more dishes for lunch, and one or more dishes for dinner. The exchanging the elements may be randomly performed, e.g., a number of the meal plans may be randomly determined, then a randomly number of elements in these meal plans are exchanged. For example, the staples for lunch in a first meal plan is exchanged with the staples for lunch in a second meal plan, and the breakfast menu in a third meal plan is exchanged with the breakfast menu in the first meal plan. In this step, new (i.e., second initial) meal plans are generated based on the previous (i.e., the first initial) meal plans.

In step 204, a score for each second initial meal plans is calculated, wherein the new score may be noted as Yi for the i-th second initial meal plan. The score calculating method may be the same as the calculating method disclosed for step 104 in FIG. 1A/1B and step 202. In addition, if one, some or all the scores of Yi meet the condition equal to or higher than the first predetermined threshold, the second initial meal plan may be directly outputted as the second subset of the meal plans and the later steps in FIG. 2 may not be performed. Otherwise, if one, some or all the scores of Yi is lower than the first predetermined threshold, the later steps may be performed.

In step 205, the evolution meal plan set is updated by, if Yi>=Xi, replacing the i-th first initial meal plan by the i-th second initial meal plan in the evolution meal plan set. In this way, the higher score meal plans may be included in the evolution meal plan set.

In step 206, steps 202 to 205 are performed iteratively. The iterations may end if the iterations have been performed already more than a predetermined number, and/or until a part of or all meal plans in the evolution meal plan set have scores equal to or higher than a first predetermined threshold. For example, if one score amongst the meal plans equals to or is higher than the first predetermined threshold, the iterations may end; or only if all scores of the meal plans equal to or are higher than the first predetermined threshold, the iterations may end.

In step 207, the evolution meal plan set is outputted as the second subset of meal plans.

With the method in FIG. 2, meal plans with high scores (e.g., according to the specific diet information of the user) are included in second subset, and at the same time the meal plans in the second subset have been randomly generated (e.g., by step 203) such that the same meal plans will be always suggested to the same user.

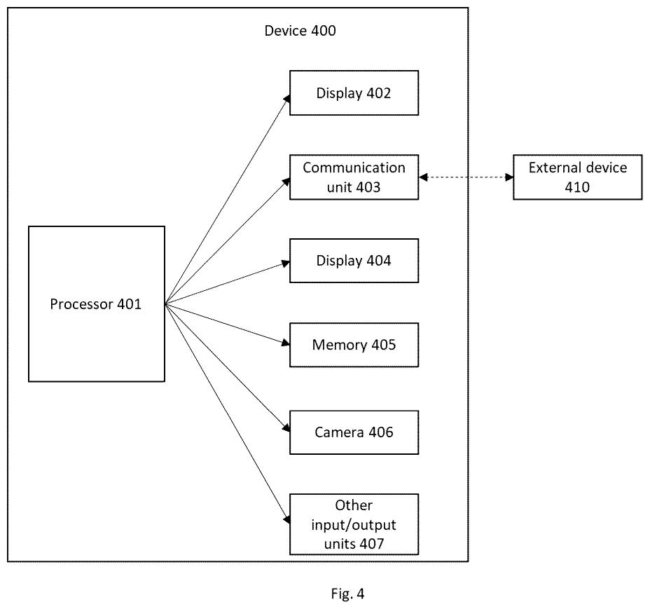

FIG. 4 shows a device 400, e.g., a mobile phone, tablet, laptop, desktop, smart watch, a TV, etc., to perform the present invention.

The device 400 may comprise a processor 401, a display 402, a communication unit 403, a memory 405, a camera 406 and other input/output units 407.

The processor 401 is configured to perform the program/instructions stored in the memory 405, e.g., via controlling other components such as the display 402, the communication unit 403, the memory 405, the camera 406 and other input/output units 407.

The displayed 402 may be controlled by the processor 401 to perform the all the displaying function (and input function if it is a touch screen) in the present invention such as in step 101.

The communication unit 403 may be controlled by the processor 401 to perform all communication function in the present invention. For example, if an external device 410 (e.g., an sever) is used to perform some functions in the steps of FIG. 1A/1B and/or FIG. 2 (e.g., step 101 may be performed on the user device 400 and the final meal plan suggestion may be displayed on the user device 400 as well, but other steps in FIG. 1A/1B and FIG. 2 may be performed on the external device 410, e.g., a server; or all the steps may be performed on the user device 400; or a part of the steps is performed on the user device 400 and the remaining part of the steps is performed on at least one or more external devices 410), messages may be communicated via the communication unit 403. Optionally, the databases used in the present invention may be stored in the external device 410 or the user device 400, e.g., the look up tables for the ingredient scores, the original meal plan sets, the weekly rules, the daily rules, the gene rules, the preference rules, the diet information of the user, etc.

The memory 405 may be configured to store the instructions to perform the methods of the present invention. For example, the look up tables for the ingredient scores, the original meal plan sets, the weekly rules, the daily rules, the gene rules, the preference rules, the diet information of the user, or other necessary information, may also be stored in the memory 405. The device may provide at least one entry for the user to check/overviewing these data.

The camera 406 is configured to capture images, which is optional in the present invention.

The other input/output units 407 may be configured to perform other input/output functions of the present invention, for example, to receive user input for the diet information and output the suggested meal plan to the user.

In the present invention, at least a part of the device (e.g., FIG. 4) or method (e.g., FIG. 1A/1B, FIG. 2 and/or FIG. 3 as computer implemented algorithms) may be implemented as instructions stored in a non-transitory computer-readable storage medium, e.g., in the form of a program module, a piece of software, a mobile app, and/or other forms. The instructions, when executed by a processor (e.g., the processor 401), may enable the processor to carry out a corresponding function according to the present invention. The non-transitory computer-readable storage medium may be the memory 405.

The present invention comprises a method, performed by an electronic device, for suggesting meal plans, comprising, obtaining diet information of a user; forming a first subset of meal plans from a predetermined meal plan set according to the diet information; generating a second subset of meal plans from the first subset of meal plans via an evolution algorithm; calculating a score for each meal plan in the second subset of meal plans; ranking meal plans in the second subset of meal plans, wherein the score for each meal plan is calculated based on the diet information and one or more elements comprised in each meal plan.

The above method may further comprise, displaying a number of top ranked meal plans and/or receiving an input from a user to select a meal plan from the displayed meal plans; or suggesting a meal plan with a highest score from the second subset of meal plans.

The above method may further comprise receiving an input from a user to change a first meal plan within the top ranked meal plans, and suggesting a second meal plan based on the first meal plan; or, receiving an input from the user to change at least one element in the first meal plan, and replacing the at least one element in the first meal plan with at least one different element to form a second meal plan.

In the above method, the suggesting of the second meal plan may be further based on at least a similarity between the first and the second meal plan, and the diet information, and/or the forming of the second meal plan may be further based on at least a similarity between the at least one element to be replaced and the at least one different element, and the diet information.

The above method may further comprise, determining at least one intake target for each food category, each macronutrient, and/or each micronutrient, according to the diet information, wherein each food category, each macronutrient and each micronutrient are predetermined.

In the above method, the one or more elements in each meal plan may comprise at least one of a breakfast menu, one or more staples for lunch, one or more staples for dinner, an extra meal menu, one or more dishes for lunch, and one or more dishes for dinner.

In the above method, the diet information may comprise at least one of allergens, restricted ingredients, gene/DNA information, a number of meals for a day, height, weight, gender, active level, location information, diet preference, diet history data, user pattern data, family history information, and stage of the user, for example the stage being pre-pregnancy, pregnancy, puerperium, or lactation.

In the above method, the gene/DNA information of rs1801133 may comprise types of GG, AG and AA, and folate needs for different types are GG>AG>AA.

In the above method the forming of the first subset of meal plans may comprise removing zero, one or more meal plans from the predetermined meal plan set which comprise the allergens and/or the restricted ingredients and/or removing meal plans from the predetermined meal plan set which may have ingredients other than existing meal preparing ingredients.

In the above method, the evolution algorithm may comprise,
a. forming an evolution meal plan set by selecting a number N of first initial meal plans;
b. calculating a score for each first initial meal plan, the score being Xi for the i-th first initial meal plan;
c. exchanging one or more elements between two or more first initial meal plans to form second initial meal plans;
d. calculating a score for each second initial meal plans, the score being Yi for the i-th second initial meal plan;
e. updating the evolution meal plan set by, if Yi>=Xi, replacing the i-th first initial meal plan by the i-th second initial meal plan in the evolution meal plan set;
f. performing steps b to e for a predetermined iterations and/or until a part of or all meal plans in the evolution meal plan set having scores equal to or higher than a first predetermined threshold;
g. outputting the evolution meal plan set as the second subset of meal plans.

In the above method, if the score Xi for one, some or all first initial meal plan equals to or is higher than the first predetermined threshold, the N first initial meal plans may be outputted as the second subset of meal plans, and steps c to g are not performed.

In the above method, if the score Yi for one, some or all second initial meal plan equals to or is higher than the first predetermined threshold, the N second initial meal plans may be outputted as the second subset of meal plans, and steps f and g are not performed.

In the above method, steps e to g may be only performed if the score Yi for one, some or all second initial meal plan is lower than the first predetermined threshold.

In the above method, the forming of the first subset of meal plans the and generating of the second subset of meal plans may be performed iteratively until a part or all of meal plans in the second subset of meal plans have scores higher than or equal to a second predetermined threshold.

In the above method, the forming of the first subset of meal plans and generating of the second subset of meal plans may be performed more than predetermined iterations, the selecting of the meal plan from the second subset of meal plans may be performed even if none in the second subset of meal plans has a score higher than or equal to the second predetermined threshold.

In the above method, a score of a meal plan may be calculated based on at least two score parts, and/or a score part being an ingredient score, a weekly score, a daily score, a gene score or a preference score.

In the above method, the ingredient score may be calculated based on at least one of a total food category score, a total macronutrient score, and a total micronutrient score, and/or the total food category score may be a sum of individual food category scores, and an individual food category score may be calculated according to mass of a food category comprised in the meal plan and intake target of the food category, for example, the food category being at least one of grain, cereal, diary, fruit, vegetable, soy products, nuts, sweets, water, meat, fish and alternatives, the total macronutrient score may be a sum of individual macronutrient scores, and an individual macronutrient score may be obtained according to mass of a macronutrient comprised in the meal plan and intake target of the macronutrient, and/or the total micronutrient score may be a sum of individual micronutrient scores, and an individual micronutrient score may be obtained according to mass of a micronutrient comprised in the meal plan and intake target of the micronutrient.

In the above method, the weekly score may be based on weekly rules concerning meal plans in a week.

In the above method, the daily score may be based on daily rules concerning a one-day meal plan.

In the above method, the gene score may be based on gene rules concerning diet requirements according to gene of the user.

In the above method, the preference score may be based on preference rules concerning diet preferences of the user.

In the above method, when calculating the score of the meal plan, each score part may be assigned with a weight value.

In the above method, the weight values for the score parts may be determined by:
determining priorities for the score parts;
assigning initial weight values to the score parts, a score part with a higher priority being assigned with an initial higher weight value;
selecting a test subset of meal plans from the predetermined meal plan set, e.g., a part of all meal plans in the predetermined meal plan set;
calculating scores for score parts based on initial weight values assigned to the score parts;
if one or more of the calculated scores satisfy at least one certain condition, determining that the initial weight values are final weight values, otherwise, iteratively calculating the scores based on at least one of assigning different initial weight values to the score parts and selecting the test subset.

In the above method, the at least one certain condition may comprise at least one of a percentage, of calculated scores that are higher than a certain score threshold, is higher than a certain percentage threshold, and calculated scores calculated based on the initial weight values are with a predetermined error range of predetermined reference scores.

In the above method, the determining of the intake target may be further based on standard nutrient recommendations according to the diet information of the user.

In the above method, the determining of the intake target may be further based on biometric information, microbiome information, and/or blood glucose information of the user.

The present invention may comprise an apparatus comprising at least one processor, wherein the at least one processor is configured to perform the above method.

The present invention may comprise a storage medium storing computer instructions, where the instructions are configured to control at least one processor to perform the above method.

The present invention may comprise a method, performed by an electronic device, for suggesting meal plans, comprising, determining a first element in the first meal plan to be replaced and a first nutritional vector of the first element, obtaining a candidate set of elements, obtaining candidate nutritional vectors for some or all the candidate elements in the candidate set, determining each similarity score based on each obtained candidate nutritional vector and the first nutritional vector, obtaining a second meal plan based on a second element which has a highest similarly score, wherein the first element in the first meal plan is replaced by the second element to form the second meal plan.

The method may further comprise displaying a number of meal plans and/or receiving an input from a user to select the first meal plan from the displayed meal plans.

More than one first element in the first meal plan may be replaced by more than one second element respectively.

The first meal plan and/or the first element may be determined according to a user input, or the first meal plan and/or the first element may be determined according to at least one predetermined condition.

The predetermined condition may include at least one of that if a same meal plan and/or element has been suggested in the last predetermined number of days, the same meal plan and/or element is determined as the first meal plan and/or element; if a meal plan and/or element includes ingredient that is not available, then they are determined as the first meal plan and/or element; and if a further user input is received which conflicts with a meal plan and/or element, then they are determined as the first meal plan and/or element, wherein the further user input may include updated diet information.

The similarity score between two elements may be calculated based on the below equation:

$$S ( A , B ) = \frac{A \cdot B}{‖ A ‖ ‖ B ‖} = \frac{{\sum }_{i = 1}^{n} {A}_{i} {B}_{i}}{\sqrt{{\sum }_{i = 1}^{n} {A}_{i}^{2}} \sqrt{{\sum }_{i = 1}^{n} {B}_{i}^{2}}}$$

wherein, A and B are the nutritional vectors of the two elements respectively, A is <A₁, A₂, . . . Aₙ>, B is <B₁, B₂, . . . Bₙ>, and S is the similarity score.

A nutritional vector of an element may include four values representing total energy, total protein, total fat, and total carbohydrate in that element, by weight or volume.

Aₙ element in a meal plan may be one of a breakfast menu, one or more staples for lunch, one or more staples for dinner, an extra meal menu, one or more dishes for lunch, and one or more dishes for dinner.

The diet information may comprise at least one of allergens, restricted ingredients, gene/DNA information, a number of meals for a day, height, weight, gender, active level, location information, diet preference, diet history data, user pattern data, family history information, and stage of the user, for example the stage being pre-pregnancy, pregnancy, puerperium, or lactation.

The gene/DNA information of rs1801133 may comprise types of GG, AG and AA, and folate needs for different types are GG>AG>AA.

The present invention may comprise an apparatus comprising at least one processor, wherein the at least one processor is configured to perform any of above methods.

The present invention may comprise a storage medium storing computer instructions, where the instructions are configured to control at least one processor to perform any of the above methods.

1. A method, performed by an electronic device, for suggesting meal plans, comprising,
determining a first element in the first meal plan to be replaced and a first nutritional vector of the first element,
obtaining a candidate set of elements,
obtaining candidate nutritional vectors for some or all the candidate elements in the candidate set,
determining each similarity score based on each obtained candidate nutritional vector and the first nutritional vector,
obtaining a second meal plan based on a second element which has a highest similarly score,
wherein the first element in the first meal plan is replaced by the second element to form the second meal plan.

2. The method of claim 1, further comprises, displaying a number of meal plans and/or receiving an input from a user to select the first meal plan from the displayed meal plans.

3. The method in any of the preceding claims, wherein more than one first element in the first meal plan is replaced by more than one second element respectively.

4. The method in any of the preceding claims, wherein the first meal plan and/or the first element is determined according to a user input, or the first meal plan and/or the first element is determined according to at least one predetermined condition.

5. The method in claim 4, wherein the predetermined condition includes at least one of that if a same meal plan and/or element has been suggested in the last predetermined number of days, the same meal plan and/or element is determined as the first meal plan and/or element; if a meal plan and/or element includes ingredient that is not available, then they are determined as the first meal plan and/or element; and if a further user input is received which conflicts with a meal plan and/or element, then they are determined as the first meal plan and/or element, wherein the further user input includes updated diet information.

6. The method in any of the preceding claims, wherein the similarity score between two elements is calculated based on the below equation:
$$S ( A , B ) = \frac{A \cdot B}{‖ A ‖ ‖ B ‖} = \frac{{\sum }_{i = 1}^{n} {A}_{i} {B}_{i}}{\sqrt{{\sum }_{i = 1}^{n} {A}_{i}^{2}} \sqrt{{\sum }_{i = 1}^{n} {B}_{i}^{2}}}$$
wherein, A and B are the nutritional vectors of the two elements respectively, A is <A₁, A₂, . . . Aₙ>, B is <B₁, B₂, . . . Bₙ>, and S is the similarity score.

7. The method in any of the preceding claims, wherein a nutritional vector of an element includes four values representing total energy, total protein, total fat, and total carbohydrate in that element, by weight or volume.

8. The method in any of the preceding claims, wherein an element in a meal plan is one of a breakfast menu, one or more staples for lunch, one or more staples for dinner, an extra meal menu, one or more dishes for lunch, and one or more dishes for dinner.

9. The method in any of the preceding claims, wherein the diet information comprises at least one of allergens, restricted ingredients, gene/DNA information, a number of meals for a day, height, weight, gender, active level, location information, diet preference, diet history data, user pattern data, family history information, and stage of the user, for example the stage being pre-pregnancy, pregnancy, puerperium, or lactation.

10. The method of claim 10, wherein the gene/DNA information of rs1801133 comprises types of GG, AG and AA, and folate needs for different types are GG>AG>AA.

11. An apparatus comprising at least one processor, wherein the at least one processor is configured to perform any of claims 1 to 10.

12. A storage medium storing computer instructions, where the instructions are configured to control at least one processor to perform any of claims 1 to 10.
